(19) United States
(12) Patent Application Publication
(10) Pub. No.: US 2020/0129119 A1
(43) Pub. Date: Apr. 30, 2020

(54) SYSTEMS AND METHODS FOR EVALUATING EFFECTS OF TRANSCRANIAL NEUROSTIMULATION

(71) Applicant: NEUROELECTRICS CORPORATION, Cambridge, MA (US)

(72) Inventors: Giulio RUFFINI, Cambridge, MA (US); Ricardo SALVADOR, (ES)

(21) Appl. No.: 16/605,723

(22) PCT Filed: Apr. 18, 2018
(86) PCT No.: PCT/US2018/028099

§ 371 Date: Oct. 16, 2019

Related U.S. Application Data
(60) Provisional application No. 62/486,743, filed on Apr. 18, 2017.

(51) Int. Cl. A61B 5/00; A61N 1/04; A61N 1/36; A61B 5/0484; A61B 5/04; A61B 6/03

(52) U.S. Cl. CPC A61B 5/4848; A61N 1/0456; A61N 1/36034; A61B 6/037; A61B 5/04009; A61N 1/36025; A61N 1/36003; A61B 5/0484

(57) ABSTRACT

The present invention provides methods for evaluating the effects of transcranial neurostimulation, including techniques for sham stimulation to provide effective subject and/or operator blinding.

### CROSS-REFERENCE TO RELATED APPLICATIONS

This application claims priority to U.S. Provisional Patent Application No. 62/486,743, filed Apr. 18, 2017. The entire content of the aforementioned patent application is incorporated herein by reference.

### FIELD OF THE INVENTION

The present invention provides methods for evaluating the effects of transcranial current neurostimulation, including techniques for sham stimulation that provide effective subject and/or operator blinding.

### BACKGROUND

Transcranial current stimulation (tCS) is a form of non-invasive neurostimulation that involves electrodes placed on the scalp to deliver weak currents to one or more target brain region(s). Transcranial current stimulation includes a family of related noninvasive techniques such as direct (tDCS), alternating (tACS), and random noise current stimulation (tRNS). tCS is being investigated for treating and/or improving performance in subjects having neurodegenerative or neurological disease, including dementia, epilepsy, tremor, mild cognitive impairment, depression, chronic pain and migraine, among others. However, in a controlled, blinded study where a sham condition is needed as control, many subjects experienced with tCS can detect the sham stimulation (e.g., non-stimulation), making it difficult to control for placebo effects. Thus, methods are desirable to provide sham neurostimulation that is less likely to be detected by subjects in a controlled investigation or during a clinical intervention. In addition, it is also important to blind the operators (double-blinding), so that they don't inadvertently influence the experimental outcome.

### SUMMARY OF THE INVENTION

The present invention provides systems and methods for evaluating effects of tCS in a subject or subject population. In various embodiments, the invention involves providing neurostimulation to a subject or group of subjects using an electrode montage and currents designed to produce skin sensation, but with shallow electric fields having minimal effects in the cortex or a specific cortical area due to a rapid intensity decrease of amplitude with depth.

In various embodiments, the methods comprise stimulating one or more cortical targets of a first subject or group of subjects with transcranial neurostimulation using a first selected montage of electrodes (M1). The montage M1 may be selected, for example, to stimulate a plurality of targets, including cortical targets, deeper targets in the brain, and/or functional networks. The method further involves providing a second subject or group of subjects with a second selected montage (M2), which optionally employs the same electrode number, type, and/or positions as M1, but using currents designed to produce skin sensation with transcranially-generated shallow electrical fields with minimal effects in the cortex or in specific cortical areas. In various embodiments, the effects of neurostimulation of the first subject or group are compared with one or more effects of neurostimulation of the second subject or group. In some embodiments, the first subject or group of subjects is the same as the second subject or group of subjects, with neurostimulation with M1 and M2 being provided at different time points. In these embodiments, sham stimulation with M2 can provide a baseline for evaluating the effects of active stimulation with M1. In various embodiments, M1 and M2 generate similar skin sensation such that subjects cannot distinguish the active neurostimulation and sham stimulation.

In various embodiments, the transcranial neurostimulation is selected from transcranial direct current stimulation (tDCS), transcranial alternating current stimulation (tACS), transcranial random noise stimulation (tRNS), or any other form of transcranial current brain stimulation (tCS). In some embodiments, the neurostimulation comprises excitatory, inhibitory, or neutral stimulation.

In some embodiments, M1 is identified by calculating optimal currents, numbers, and locations of electrodes for globally stimulating at once one or more cortical targets. In some embodiments, the optimal number and locations of electrodes are determined using a genetic algorithm, to identify an electrode montage with a specified fitness for stimulating the one or more cortical targets. In various embodiments, the method involves the use of a target map, optionally generated from brain activity data and/or neuroimaging data, and which specifies desired values for the electric field and weights assigned to target regions to stimulate at once the one or more cortical targets.

In various embodiments, M1 and M2 generate similar scalp sensations, such as an itch sensation, using one or more of a configured multielectrode array, selection of electrode sizes, selection of electrode shapes, and selection of an electrode interface.

In some embodiments, M2 uses the same electrode number, locations, shapes, and/or sizes as M1, but currents are determined for M2 to generate shallow electrical fields with minimal effects in the cortex or in specific cortical areas. In some embodiments, at least one electrode of M2 has a current of at least 500 μA to generate skin sensation. In some embodiments, one or more electrodes in M1 have a different location in M2, but sensations are perceived similarly.

Methods of the invention may be used to evaluate neurostimulation in various populations of diseased or healthy subjects, including for evaluating treatment of a neurological disorder, including neurodegenerative disease, dementia, epilepsy, tremor, obsessive-compulsive disorder, dystonia, stroke, depression, Tourette syndrome, migraine, and chronic pain, among others. In various embodiments, methods of the invention are useful for evaluating the effects of neurostimulation in improving one or more of memory, cognition, and/or motor functions in healthy and/or diseased individuals.

Other aspects and embodiments of the invention will be apparent to one of ordinary skill in the art in view of the following detailed description.

### DETAILED DESCRIPTION

The present invention provides systems and methods for evaluating effects of tCS in a subject or subject population. In various embodiments, the invention involves providing sham neurostimulation to a subject or group of subjects using an electrode montage with currents designed to produce skin sensation, but with shallow electric fields. The shallow electric fields are weak in the cortex area of interest (which may be the entire cortex), with local or target-average magnitudes smaller than, e.g., about 0.05 V/m, and thus have minimal physiological effects in the cortex. In the various embodiments, the invention provides effective subject and/or operator blinding in controlled studies involving tCS. In some embodiments, the invention is employed during a clinical intervention, to confirm that the patient is responding to neurostimulation treatment rather than to placebo.

tCS is a noninvasive brain stimulation technique in which weak, constant or slowly varying electrical currents are applied to the brain through the scalp. tCS includes a family of related noninvasive techniques including direct (tDCS), alternating (tACS) and random noise current stimulation (tRNS). These techniques use scalp electrodes with electrode current intensity to area ratios of about 0.3 to about 5 A/m² at low frequencies resulting in weak but physiologically relevant electric fields in the brain, with amplitudes of about 0.2 to 2V/m.

In various embodiments, the transcranial neurostimulation (tCS) is transcranial direct current stimulation (tDCS), transcranial alternating current stimulation (tACS), transcranial random noise stimulation (tRNS), or any other form of tCS. In an embodiment, the present methods involve transcranial direct current stimulation (tDCS). In some embodiments, the tCS (for active stimulation) provides an electrode current intensity to area ratio of about 0.3 to about 5 A/m² through the scalp. In some embodiments, the tCS (for active neurostimulation) is delivered at low frequencies of less than about 5 kHz, or less than about 4 kHz, or less than about 3 kHz, or less than about 2 kHz, or less than about 1 kHz. In some embodiments, the tCS for active neurostimulation results in weak electric fields in the brain with amplitudes of about 0.2 to about 2 V/m.

In various embodiments, the present invention comprises providing neurostimulation to an experimental group (e.g., “active neurostimulation”). Active neurostimulation involves stimulating one or more cortical targets of a first subject or group of subjects (the experimental group) with transcranial neurostimulation using a first selected montage of electrodes (M1). As used herein, the term “electrode montage” refers to a configuration of electrodes comprising the number, location, and type of electrodes, as well as electrode current. Once parameters are determined for the experimental group (e.g., montage M1), the method further involves providing a second selected montage (M2) to provide a sham neurostimulation to a control subject or group. The objective of the sham stimulation is to produce skin sensations that are perceived by the subject to be comparable to skin sensations resulting from active neurostimulation, but generate shallow electrical fields with minimal effects in the cortex or in specific cortical areas. In various embodiments, at least one electrode in M2 has a high current of, for example, at least 500 μA to generate skin sensation. The desired current needed to generate skin sensation will depend in part on electrode shape and size. In some embodiments, the electrode locations, electrode shapes, and electrode sizes for M1 and M2 are the same, providing optimal subject and operator blinding. In some embodiments, one or more electrode locations, or electrode shapes, or electrode sizes for M2 can differ from M1, as long as scalp sensations are similar.

Skin sensations can be produced in M1 and M2 by configuring montages based on having at least one electrode with a sufficient current to generate skin sensation, or by the use of one or more electrodes having a size and/or shape that is likely to generate skin sensation. For example, small electrodes of from about 0.5 cm to about 0.2 cm radius are more likely to generate skin sensation (as compared to large electrodes of at least about 1 cm radius). Current intensity being constant, small electrodes produce stronger skin sensations but similar cortical electrical field amplitudes, as compared to larger electrodes. Thus, maintaining one or more small electrodes in both M1 and M2 (optionally at fixed positions), and optionally in a montage that also comprises one or more large electrodes, will induce a scalp sensation that blinds active and sham stimulation. Alternatively, or in addition, using a montage (M1 and M2) that comprise one or more non-round electrodes, such as square, triangular, or star shaped, will induce a similar scalp sensation, since round electrodes are reported to be less itchy than electrodes that comprise corners. In these or other embodiments, M1 and/or M2 can employ an electrochemical interface, such as a conduction gel, that generates an itch, e.g., due to changes such as pH in the electrode interface.

In various embodiments, the effects of neurostimulation of the first subject or group are compared with one or more effects of neurostimulation of the second subject or group to account for any placebo effects of the neurostimulation. The method can be employed in the context of a controlled study with experimental and control cohorts, or alternatively, in the context of optimizing clinical intervention by establishing a baseline for the subject using sham stimulation to control for placebo effects.

Conventionally, sham neurostimulation is conducted in controls by ramping up and down currents after some 10 to 60 seconds, for example, since it is observed that subjects report skin sensations such as tingling and itch due to tCS primarily at the beginning of stimulation. However, it has been reported that some subjects, especially those experienced with neurostimulation, may still distinguish the sham condition. Furthermore, even if successful at blinding some subjects, the fact that there is no current applied during most of the period in conventional sham stimulation introduces the problem that the observed differences between sham and active conditions may be due to differences in peripheral nervous stimulation at the scalp, which may itself indirectly influence cortical activity. In contrast, shallow electric fields shunted through the scalp and cerebrospinal fluid (CSF) in accordance with M2 will result in small or very limited electric fields in the cortex while still stimulating the peripheral nervous system (PNS). Therefore, in certain embodiments the invention provides means to differentiate between cortical and possible PNS effects. This is an additional benefit of this disclosure beyond the fact that, given the skin sensations generated by M2, even experienced subjects will not distinguish the conditions. In some embodiments, the sham current stimulation with M2 in accordance with this disclosure is provided for the same or similar duration and intensity as active stimulation (e.g., with M1), thereby differentiating PNS from cortical effects.

The present invention allows for controlled neurostimulation, including for clinical studies and clinical intervention. For example, the invention provides for active neurostimulation of an experimental subject or group. The experimental subject or group is referred to herein as “the first group.” The invention further provides for sham neurostimulation of a control subject or group. The control subject or group is referred to herein as “the second group.” In various embodiments, the first and/or second group comprises at least 1 subject, but may comprise at least about 10 subjects, at least about 20 subjects, at least about 50 subjects, or at least about 100 subjects. While the first group of subjects may be different than the second group of subjects, in some embodiments, the first group is the same as the second group of subjects with active neurostimulation and sham neurostimulation provided at different time points, optionally in random order. In some embodiments, sham neurostimulation is provided during a clinical intervention, to distinguish effective treatment with neurostimulation from placebo effects. In some embodiments, stimulation with M2 is provided for one or more sessions and used as a baseline for evaluating stimulation with M1.

Transcranial neurostimulation is provided to the first group of subject(s) using a first selected montage of electrodes (M1), to provide excitatory, inhibitory or neutral stimulation. In various embodiments, M1 is configured to provide transcranial stimulation for the treatment of a particular neurological or psychiatric disorder or cognitive deficiency, as described elsewhere herein. In various embodiments, M1 is configured to provide transcranial stimulation personalized for a particular subject based on a brain monitoring technology. In some embodiments, M1 is configured to stimulate one or more cortical targets or deep brain targets. In some embodiments, M1 is configured to stimulate a functional network.

In various embodiments, currents, electrode locations, and electrode numbers are determined for M1 using a realistic head model with electric field modeling. See Miranda et al., “The electric field in the cortex during transcranial current stimulation”, Neuroimage 70, 45-58 (2013). In some embodiments, the realistic head model is a multilayer finite element model of a realistic head that may be either generic or specific to a patient. In such embodiments, tissue boundaries are derived from MR images (scalp, skull, cerebrospinal fluid (CSF) including ventricles, Grey Matter and White Matter), and the finite element method is used to calculate the electric potential in the head, subject to the appropriate boundary conditions. Tissues are assumed to be uniform and possibly isotropic (although anisotropy can also be modeled), and values for their electric conductivity are taken from the literature or measured using techniques such as electrical impedance tomography (EIT) or Magnetic Resonance Electrical Impedance Tomography (MREIT).

In some embodiments, one or more cortical targets are identified from brain activity data and/or neuroimaging data from one or more subjects. In some embodiments, the brain activity data and/or neuroimaging data are obtained with brain monitoring technology selected from one or more of functional MRI (fMRI), resting-state functional connectivity MRI (rs-fcMRI), positron emission tomography (PET), electroencephalography (EEG), magnetoencephalography (MEG), Single-photon emission computed tomography (SPECT), functional near-infrared spectroscopy (fNIRS), and a combination thereof.

Montage M1 comprises a specified number of electrodes, specified location of electrodes, as well as specified currents. Determination of number and location of electrodes and optimal currents to stimulate multiple targets at once is described in U.S. Pat. No. 9,694,178, the entire disclosure of which is hereby incorporated by reference. Generally, the optimization of currents, electrode locations and electrode numbers can employ extended, weighted cortical pattern target maps based on brain activity data and/or neuroimaging data. The target maps define desired values for the electric field at multiple points for stimulation. Targets can be defined based on a coordinate system relative to the cortical surface, with target values for normal and/or tangential components of electric field. The process can use algorithms to optimize currents as well as the number and location of electrodes given appropriate constraints, such as the maximum current at any electrode and the maximum total injected current. For example, M1 can be determined using a target map of a cortical surface specifying desired values for the electric field at each point. Further, determination of M1 can employ a weight map providing the degree of relative importance of each location in the target map, and a set of constraints on the number of electrodes and their currents. In some embodiments, the weighted target map of the cortical surface is generated by prioritizing the areas in the target map for optimization purposes. For example, a higher weight is given to those brain areas considered to be more important for the particular application of neurostimulation.

In various embodiments, M1 is determined by providing a signed (i.e., positive or negative) target map of electric field characteristics of the brain's cortex. In some embodiments, the target map can be a user-defined area or areas in the cortical surface. In some embodiments, the target maps can be defined ad-hoc by the user, or they can stem from, e.g., fMRI, PET, MEG or EEG data. In the latter case, techniques such as bandpass filtering and cortical mapping (a variation of EEG tomography where the generating dipoles are constrained on the cortical surface) may be used to generate target maps. In some embodiments, rs-fcMRI seed correlation t-test or statistical significance maps (“t-maps”) may be employed as they can provide links to deep regions not easily accessible by non-invasive stimulation techniques.

In some embodiments, the target map includes one or more cortical targets, wherein one or more cortical targets may be localized (i.e., well-delineated isolated target locations in the cortex). In some embodiments, the target map includes one or more cortical targets that are continuously varying and/or spatially extended. In some embodiments, the targets are static, while in other embodiments, the targets are dynamic (e.g., time varying targets). In such embodiments, transcranial neurostimulation may involve a determination of temporal features in addition to calculation of optimal currents, optimal electrode locations and/or optimal electrode numbers.

In some embodiments, the one or more cortical targets are final targets. In these or other embodiments, one or more cortical targets are intermediary targets whose spatially extension patterns indirectly affect, via neuronal interaction, cortical or deeper targets in the brain. In some embodiments, the targets are deep brain targets. In some embodiments, the targets are selected so as to stimulate a functional brain network. In such embodiments, stimulation not only directly targets the affected region, but the entire cortex via functional networks. In some embodiments, one or more functional networks are identified by rs-fcMRI.

In some embodiments, the calculation of currents and electrode locations is performed under constraints regarding maximal electrode number, maximal or minimal current at each electrode, and the total current injected into the brain by all electrodes at any time. In some embodiments, the calculations are performed under additional constraints including holding the current in an electrode at a constant fixed value.

In some embodiments, the target map of the invention may include zero values (areas in which no effect of stimulation is wanted) as well non-zero values associated with specific targets. In some embodiments, in order to increase focality for a cortical target, the calculation of currents and electrode locations for M1 generates zero or near zero electric field values for those electrodes surrounding said cortical target of increased focality.

In some embodiments, the calculation of currents for M1 uses least squares. In an embodiment, the present method comprises using constrained least squares to optimize current intensities. Exemplary methods for current optimization are described in U.S. Pat. No. 9,433,785, the entire disclosure of which is hereby incorporated by reference.

In some embodiments, the calculation of optimal electrode locations and/or optimal electrode numbers employs a genetic algorithm. Genetic algorithms are described, for example, in U.S. Pat. No. 9,694,178, the entire disclosure of which is hereby incorporated by reference. The genetic algorithm can be based on the definition of a solution by a “DNA” binary string (in this case of dimension N−1) specifying the electrode locations and number, and currents, and may employ as an optimization function the least squares error, i.e., the one with the best possible current configuration for the chosen electrode locations. Cross-over and mutation functions are defined to ensure that the offspring of solutions do not violate the constraint of maximal number of electrodes in the solution. Once a DNA string is specified (i.e., a particular montage), its fitness can be computed by inverting the solution for that particular montage. Solutions with more than the maximal number of electrodes desired are penalized strongly. The genetic algorithms with specifically designed fitness, cross-over and mutation functions, converge quickly and reliably to a solution.

Once M1 is determined, either ad hoc by the user or computationally as described above, M2 can be determined for a corresponding control to provide sham neurostimulation to achieve subject and/or operator blinding. That is, target and weight maps, realistic head model and genetic algorithms are applied to obtain the optimal solution for M2. M2 has a different target map than M1 (e.g., shallow fields in at least some cortical areas), with appropriate weights, and additional constraints associated to provide for itchy electrodes, such as for example the minimal current in at least one electrode.

For example, in some embodiments, M2 includes at least one electrode with a current of at least about 500 μA, so as to generate skin sensations that are not distinguishable from active neurostimulation. The actual value will depend on factors such as electrode type and size. Generally speaking, a 500 μA current using Pistim electrodes (Neuroelectrics, Cambridge, Mass.) (Ag/AgCl 1 cm radius electrodes), generates skin sensations in most subjects. In some embodiments, M1 or M2 or both employ one or more small electrodes, or electrodes that are non-round, with a current sufficient to generate skin sensations. M2 further delivers transcranially-generated shallow electrical fields with minimal values in the cortex or in specific cortical areas. In some embodiments, the electric fields generated by M2 have minimal effects over cortical targets. These shallow electric fields shunted through the scalp and CSF will result in small or very limited electric fields in the cortex, and are physiologically inert. In some embodiments, these shallow electric fields are determined for the same electrode number, location, and/or type as used in M1, to provide both subject and operator blinding.

For example, using the same algorithm for determining M1, a suitable M2 can be determined where at least one electrode has a minimal current of, e.g., about 500 μA (or other value for the selected electrode type suitable for generating skin sensations in the subject). For example, the algorithm is asked to provide the optimal currents to match the target(s) (field equal to zero on desired cortical target(s) and on rest of the brain, with appropriate weight map). The electrode positions can be constrained to be the same as those in M1, for instance, which has the advantage of blinding the operator as well as the subject (double-blinding).

In various embodiments, the location of electrodes as used in M1 and/or M2 is arranged according to an EEG 10-20 or 10-10 system, or any other montage scheme with determined electrode positions based on a set of pre-defined locations.

In various embodiments, M1 and M2 employ the same electrode types, positions and interfaces to blind both subject and operator.

In various embodiments, the number of electrodes used in M1 and/or M2 is from 2 to 256 electrodes, and in various embodiments, employs about 2 to about 200 electrodes, about 2 to about 100 electrodes, or about 2 to about 50 electrodes, or about 2 to about 40 electrodes, or about 2 to about 20 electrodes, or about 2 to about 10 electrodes. In some embodiments, M1 and/or M2 employ from about 2 to about 8 electrodes, such as from about 4 to about 8 electrodes.

The effects of neurostimulation for the first group and the second group are compared, for example, to determine whether the neurostimulation was effective to ameliorate or prevent a condition or symptom, or to impact (e.g., improve or worsen) a neurological function. The present invention effectively deals with placebo effects of neurostimulation as well as potential PNS effects, to improve evaluation of the effects of neurostimulation for treatment of a disease or condition or for improving performance of healthy or diseased individuals.

In some embodiments, transcranial neurostimulation is provided to the first and/or second subject or group of subjects to evaluate the effects of neurostimulation on a neurodegenerative disease. Exemplary neurodegenerative diseases include, but are not limited to, Alzheimer's disease, Parkinson's disease, mild cognitive impairment, and dementia.

In some embodiments, transcranial neurostimulation is provided to the first and/or second subject or group of subjects to evaluate the effects of neurostimulation on a neurological condition. Exemplary neurological conditions include, but are not limited to, tremor, obsessive-compulsive disorder, epilepsy, dystonia, stroke, depression, Tourette syndrome, migraine, and chronic pain.

In some embodiments, transcranial neurostimulation is provided to the first and/or second subject or group of subjects to evaluate the effects of transcranial neurostimulation for improving one or more of memory, cognition, and/or motor functions in a healthy or diseased individual.

Other aspects and embodiments of the invention will be apparent to one of ordinary skill in the art in view of the present disclosure.

1. A method for evaluating effects of transcranial neurostimulation comprising:
stimulating one or more cortical targets of one or more subjects of a first group with transcranial neurostimulation using a first electrode montage (M1);
providing transcranial neurostimulation to one or more subjects of a second group with a second electrode montage (M2) including currents designed to produce skin sensation of similar intensity and duration as M1 but with transcranially-generated shallow electrical fields with minimal effects in the cortex or in a specific cortical area; and
comparing one or more effects of neurostimulation of the first group with one or more effects of neurostimulation of the second group.

2. The method of claim 1, wherein the positions of the electrodes in the second electrode montage (M2) are the same as the first electrode montage (M1).

3. The method of claim 1, wherein the positions of one or more electrodes in the second electrode montage (M2) are different from the first electrode montage (M1).

4. The method of any one of claims 1 to 3, wherein the transcranial neurostimulation is transcranial current brain stimulation (tCS) selected from transcranial direct current stimulation (tDCS), transcranial alternating current stimulation (tACS), or transcranial random noise stimulation (tRNS), or any other form of tCS.

5. The method of claim 4, wherein the transcranial current brain stimulation (tCS) delivers weak currents through the scalp at low frequencies resulting in weak electric fields in the brain.

6. The method of claim 5, wherein the tCS provides an electrode current intensity to area ratios of about 0.3-5 A/m² through the scalp.

7. The method of claim 5, wherein the tCS is delivered at a low frequency of <5 kHz.

8. The method of claim 5, wherein the tCS results weak electric fields in the brain with amplitudes of about 0.2-2 V/m.

9. The method of claim 1, wherein the transcranial neurostimulation is tDCS.

10. The method of claim 1, wherein the transcranial neurostimulation is tACS or tRNS.

11. The method of any one of claims 1 to 10, wherein said neurostimulation comprises excitatory, inhibitory, or neutral stimulation.

12. The method of any one of claims 1 to 11, wherein, for the second group, at least one electrode has a current of at least 500 μA.

13. The method of any one of claims 1 to 12, wherein M1 and M2 generate similar skin sensations.

14. The method of claim 13, wherein skin sensations are generated by at least one small electrode and/or at least one non-round electrode.

15. The method of claim 13 or 14, wherein currents with M2 and/or M1 are delivered using a conducting gel that induces skin sensation.

16. The method of any one of claims 1 to 15, wherein, in the second group, the electric field generated by the montage M2 is minimal over the cortical targets.

17. The method of any one of claims 1 to 16, wherein the electrode montage M1 is identified by calculating optimal currents, number, and location of electrodes for globally stimulating at once one or more cortical targets.

18. The method of claim 17, wherein electrode montage M2 is identified by calculating optimal currents, number, and location of electrodes for globally stimulating at once one or more cortical targets, with appropriate configuration for the desired shallow electric fields with constraints for itchy electrodes.

19. The method of claim 17, wherein M2 is configured manually.

20. The method of claim 17, wherein the cortical targets are identified from brain activity data and/or neuroimaging data.

21. The method of claim 20, wherein the brain activity data and/or neuroimaging data are obtained with brain monitoring technology selected from one or more of fMRI, rs-fcMRI, PET, EEG, MEG, SPECT and fNIRS.

22. The method of claim 20, wherein a target map is constructed from the brain activity data and/or neuroimaging data, the target map specifying desired values for the electric field for the plurality of cortical targets, the cortical targets defined by a coordinate system relative to the cortex surface.

23. The method of claim 22, wherein the target map is a weighted target map.

24. The method of claim 22 or 23, wherein the target map includes target values for normal components and/or tangential components of the respective electric field vectors.

25. The method of any one of claims 22 to 24, wherein the optimal number and locations of electrodes are determined using a genetic algorithm.

26. The method of any one of claims 17 to 25, wherein optimal currents, number, and location of electrodes is determined using a realistic head model with electric field modeling.

27. The method of any one of claims 1 to 26, wherein at least one cortical target is a final target.

28. The method of any one of claims 1 to 27, wherein one or more cortical targets are intermediary targets.

29. The method of claim 28, wherein said global stimulation stimulates a deep brain target.

30. The method of any one of claims 27 to 29, wherein targets are selected to stimulate a functional network.

31. The method of claim 30, wherein the network is identified by rs-fcMRI.

32. The method of any one of claims 17 to 31, wherein current intensities are calculated by least squares.

33. The method of any one of claims 1 to 32, wherein the plurality of cortical targets is selected for treatment of a pathology or is personalized for a subject based on a brain monitoring technology.

34. The method of any one of claims 1 to 33, wherein the electrode montage contains from 2 to 256 electrodes.

35. The method of claim 34, wherein the electrode montage contains from 2 to 8 electrodes, or contains from 4 to 8 electrodes.

36. The method of claim 34 or 35, wherein the electrode locations are arranged according to an EEG 10-20 or 10-10 system.

37. The method of any one of claims 1 to 36, wherein the first group and the second group have at least 5 subjects.

38. The method of any one of claims 1 to 36, wherein the subject(s) in the first group and the second group are the same.

39. The method of claim 38, wherein stimulation with M2 is provided for one or more sessions, followed or proceeded by stimulation with M1 for one or more sessions.

40. The method of any one of claims 1 to 39, wherein neurostimulation is provided for treatment of a neurodegenerative disease, optionally selected from Parkinson's disease, Alzheimer's disease, mild cognitive impairment, and dementia.

41. The method of any one of claims 1 to 39, wherein neurostimulation is provided for a condition selected from tremor, obsessive-compulsive disorder, epilepsy, dystonia, stroke, depression, Tourette syndrome, migraine, and chronic pain.

42. The method of any one of claims 1 to 39, wherein neurostimulation is provided for the purposes of improving memory, cognition, and/or motor function in a healthy or diseased individual.
